(19) United States
(12) Patent Application Publication
(10) Pub. No.: US 2021/0187185 A1
(43) Pub. Date: Jun. 24, 2021

(54) CANNULA FOR THE DRAINAGE OF BLOOD VESSELS

(71) Applicant: EUROSETS S.R.L., Medolla (IT)

(72) Inventors: Nicola GHELLI, Medolla (IT); Paolo FONTANILI, Medolla (IT)

(73) Assignee: EUROSETS S.R.L., Medolla (IT)

(21) Appl. No.: 17/263,092

(22) PCT Filed: Jul. 24, 2019
(86) PCT No.: PCT/IB2019/056326

§ 371 Date: Jan. 25, 2021

(30) Foreign Application Priority Data
Jul. 26, 2018 (IT) 102018000007540

(51) Int. Cl. A61M 1/36

(52) U.S. Cl. CPC A61M 1/3659; A61M 2205/0266; A61M 2205/3344

(57) ABSTRACT

A cannula for the drainage of blood vessels having a tubular body provided with a first and second ends, the second end being insertable inside a patient's blood vessel, and provided with at least one main channel extending between the ends, the tubular body being movable between a home configuration and an extended configuration, in which the diameter of said main channel is reduced with respect to the home configuration; an elastic part associated with said tubular body and adapted to counteract the displacement from the home configuration to the extended configuration; pressure detection part for detecting the pressure inside the blood vessel which are associated with the tubular body; wherein the pressure detection part comprise at least one pressure sensor associated with the tubular body in the proximity of the second end, and where the pressure sensor is arranged inside the main channel.

### TECHNICAL FIELD

The present invention relates to a cannula for drainage of blood vessels.

### BACKGROUND ART

To date, the use of cannulae for drainage of blood vessels during the extracorporeal circulation (ECC) is widespread.

The cannulae can be of the venous or arterial type depending on whether they are used for the drainage of venous or arterial blood, so as to convey it from the patient to the extracorporeal circuit and from the extracorporeal circuit to the patient, respectively.

The cannulae can also be used during various types of operations, such as e.g. laparoscopy, endoscopy and arthroscopy, to suction fluids or carry out perfusions.

The known type of cannulae do however have some drawbacks.

In particular, they do not allow medical personnel to be given any indication of their correct positioning.

As is well known, in fact, depending on the positioning of the cannula within the relative vessel, its draining capacity varies and, therefore, the flow rate of the blood to the various devices of the extracorporeal circuit or to the patients themselves.

For example, in the event of the cannula being placed “on the wall”, the blood flow rate through it is significantly reduced compared to the nominal flow rate, so it must be repositioned to achieve optimal operating conditions.

The bloodstream through the cannula can also be altered if there is a malfunction, such as an occlusion, of the extracorporeal circuit devices connected to the cannula itself.

Some cannulae for drainage of blood vessels of known type are described by US 2006/270962A1 and WO 00/12148A2.

### DESCRIPTION OF THE INVENTION

The main aim of the present invention is to devise a cannula for drainage of blood vessels that allows providing medical personnel with significant information about its placement inside the patient's vessel.

Within this aim, one object of the present invention is to provide medical personnel with immediate information about the flow rate of blood which flows through the extracorporeal circuit.

Another object of the present invention is to devise a cannula for drainage of blood vessels that allows overcoming the aforementioned drawbacks of the prior art in a simple, rational, easy, effective to use as well as cost-effective solution.

The aforementioned objects are achieved by the present cannula for drainage of blood vessels according to claim 1.

### BRIEF DESCRIPTION OF THE DRAWINGS

Further characteristics and advantages of the present invention will be more evident from the description of a preferred, but not exclusive, embodiment of a cannula for drainage of blood vessels, illustrated as an indication, but not limited to, in the attached tables of drawings in which:

FIG. 1 is an axonometric view of an aortic cannula according to the invention;

FIG. 2 is a longitudinal sectional view of the cannula of FIG. 1;

FIG. 2a is a magnification of a detail of the cannula of FIG. 2;

FIG. 3 is a sectional view of the cannula of FIG. 1 along the track plane III-III;

FIG. 4 is an axonometric view of a venous cannula according to the invention;

FIG. 5 is a longitudinal sectional view of the cannula of FIG. 4;

FIG. 5a is a magnification of a detail of the cannula of FIG. 4;

FIG. 6 is a sectional view of the cannula of FIG. 4 along the track plane VI-VI.

### EMBODIMENTS OF THE INVENTION

With particular reference to these figures, a cannula for drainage of blood vessels has been globally indicated with reference numeral 1.

The cannula 1 comprises a tubular body 2 provided with a first end 2a and with a second end 2b, the latter being intended to be inserted inside a blood vessel of a patient, and provided with at least one main channel 3 extending between the ends 2a and 2b.

More particularly, the first end 2a and the second end 2b define at least a first mouth 4 and at least a second mouth 5, respectively, for the flow of blood, where the first and the second mouth 4 and 5 are communicating with each other through the main channel 3.

The tubular body 2 is movable, for the insertion into and removal from the patient's body, between a home configuration and at least one extended configuration, in which the diameter of the main channel 3 is reduced with respect to the home configuration.

In particular, the movement of the tubular body 2 from the home configuration to the extended configuration is carried out by means of special actuating means (not shown in the figures) that can be inserted inside the main channel 3. The actuating means are, e.g., of the type of a spindle that is inserted inside the first mouth 4 and by means of which a force is exerted on the second end 2b in the direction of moving away of the second mouth 5 from the first mouth itself.

The cannula 1 then comprises elastic means 6 associated with the tubular body 2 and adapted to bring it back to the home configuration as a result of the displacement thereof to the extended configuration.

More in detail, the elastic means 6 are of the type of a weave of metal shape-memory filaments, embedded inside the tubular body 2. The elastic means 6 counteract therefore the displacement of the tubular body 2 from the home configuration to the extended configuration as a result of the force exerted by the actuating means and, once these are removed, they in turn exert a return force on the tubular body 2 to bring it back to the home configuration.

The cannula 1 also comprises pressure detection means 7 for detecting the pressure inside the patient's blood vessel which pressure detection means are associated with the tubular body 2.

According to the invention, the pressure detection means 7 comprise at least one pressure sensor 8 associated with the tubular body 2 in the proximity of the second end 2b. As can be seen from the embodiments shown in the figures, the pressure sensor 8 is arranged at at least one of the second mouths 5.

More particularly, the pressure sensor 8 is of the type of a pressure transducer, thus adapted to convert the detected pressure signal into an electrical signal.

Still according to the invention, the pressure sensor 8 is arranged inside the main channel 3.

Since, as mentioned above, the pressure sensor 8 is of the type of a pressure transducer, the pressure detection means 7 also comprise electrical connecting means 12 of the pressure sensor 8 to reading means 13, of the type of a monitor or the like. The connecting means 12 are e.g. of the type of electrical wires or optical fibers.

Appropriately, the tubular body 2 comprises at least one connecting channel 14, separate from the main channel 3 and adapted to contain the connecting means 12.

As can be seen from FIGS. 2 and 2a, and 5, 5a, the connecting channel 14 extends substantially parallel to the main channel 3 and is defined at the outer wall of the tubular body 2. The connecting channel 14 does not interfere, therefore, with the elastic means 6.

The connecting channel 14 is defined on the tubular body 2 and the pressure sensor 8 is arranged outside the connecting channel itself.

More in detail, on the tubular body 2 at least one hole 15 is defined for the passage of the connecting means 12 which protrude from the connecting channel itself, so as to allow the insertion thereof inside the main channel 3.

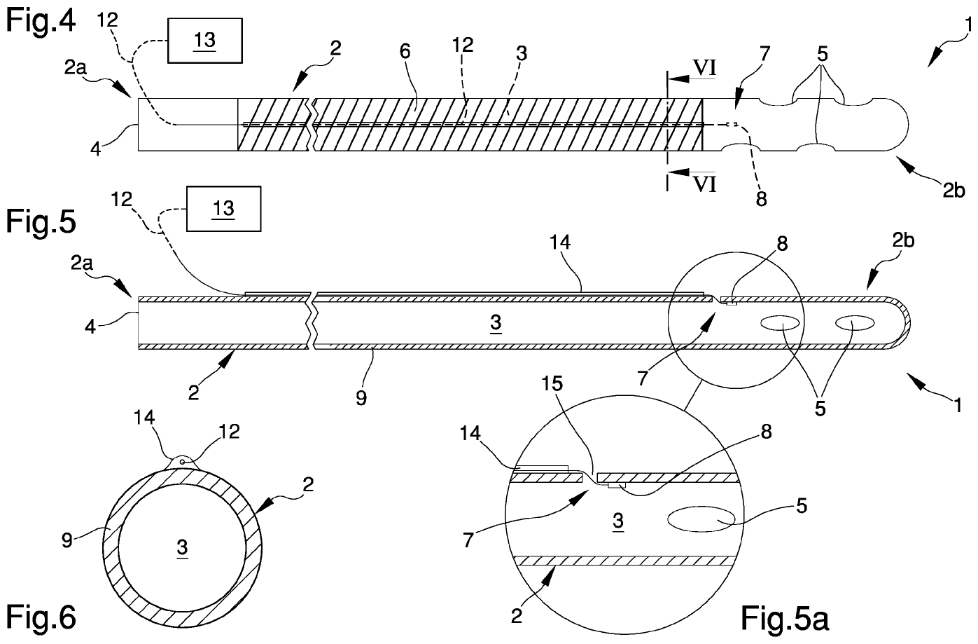

In the embodiment shown in FIGS. 4 to 6, relative to a venous cannula, that is to say, one adapted to drain the blood to be oxygenated from the patient, the tubular body 2 is made in a single body piece and comprises a plurality of second mouths 5 through which the blood flows out of the patient. In this embodiment, the pressure sensor 8 is fixed to the inner wall of the tubular body 2 and is located in the proximity of the second mouth 5 closest to the first mouth 4.

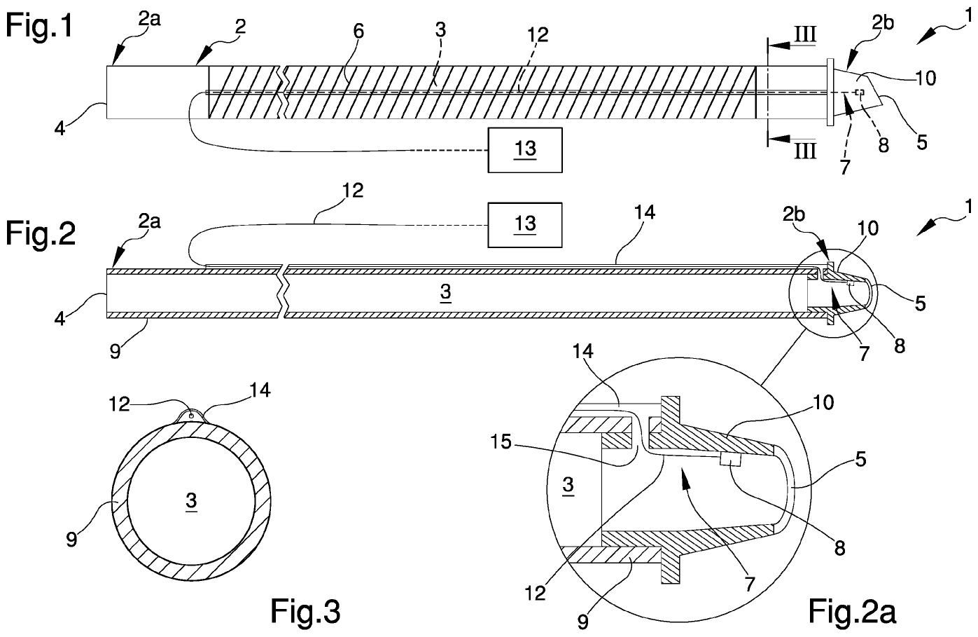

In the embodiment shown in FIGS. 1 to 4, relative to an arterial cannula, i.e. adapted to introduce the oxygenated blood into the patient's body, the tubular body 2 comprises at least one main body 9, made of a flexible material, and at least one connector 10, associated with the main body 9 and defining the second end 2b.

More in detail, the main body 9 defines the first mouth 4 and, on the opposite side, a further mouth through which the connector 10 is inserted, which in turn defines the second mouth 5. The main channel 3 is therefore delimited both by the main body 9 and the connector 10.

The elastic means 6 are associated with the main body 9.

The pressure sensor 8 is preferably associated with the connector 10.

More particularly, as shown in FIG. 2a, the pressure sensor 8 is fixed to the inner wall of the connector 10 delimiting the main channel 3.

In this embodiment, the connecting channel 14 is defined at the outer wall of the main body 9.

The pressure sensor 8 is, on the contrary, arranged outside the connecting channel 14 and the hole 15 is defined on the connector 10, in particular on its portion inserted inside the main body 9.

Preferably, the connecting means 12 and the pressure sensor 8 are fixed to the main body 9 and to the connector 10, respectively, by means of a resin.

The operation of the present invention is as follows.

Once the cannula 1 has been positioned inside the patient's vessel, the pressure measured by the pressure sensor 8 is monitored by means of the reading means 13.

In the case of a venous cannula 1, as the one shown in FIGS. 4 to 6, inserted therefore into the vena cava or the femoral vein of the patient, the detection of the suction pressure (negative pressure) of the blood allows identifying if the wall of the vessel is closing the second mouth 5 of the tubular body 2, with the additional risk of damaging the vessel itself.

In the case of an arterial cannula 1, such as the one shown in FIGS. 1 to 3, inserted therefore into the aorta artery or femoral artery of the patient, the detection of the inlet pressure (positive pressure) of the blood allows identifying whether the bloodstream is directed towards the wall of the artery, with the consequent risk of trauma of the wall itself with the possible detachment of the deposited plaques, or towards its central area.

The pressure sensor 8 therefore provides immediate and continuous pressure feedback at the second mouth 5 of the cannula 1, which allows medical personnel to identify an incorrect positioning of the cannula itself and, therefore, to prevent potential trauma to the patient.

It has in practice been ascertained that the described invention achieves the intended objects and, in particular, the fact is underlined that the cannula to which the present invention relates, allows medical personnel to easily and practically identify the positioning thereof inside the blood vessel, thus preventing the occurrence of traumatic events for the patient and the correct occurrence of extracorporeal circulation.

In particular, the use of a pressure transducer allows, given its small size, not altering the bloodstream through the cannula and, at the same time, providing immediate and reliable feedback of the pressure inside the vessel.

In addition, the presence of a connecting channel outside the main channel through which the blood flows allows minimizing the incidence of the means for detecting the pressure on the bloodstream.

1. A cannula for drainage of blood vessels including:
a tubular body provided with a first end and with a second end, the latter being intended to be inserted inside a blood vessel of a patient, and provided with at least one main channel extending between said ends, said tubular body being movable between a home configuration and at least one extended configuration, in which the diameter of said main channel is reduced with respect to the home configuration to allow it to be inserted into a patient's body;
elastic means associated with said tubular body and adapted to counteract the displacement from the home configuration to the extended configuration;
pressure detection means for detecting the pressure inside the patient's blood vessel which are associated with said tubular body;
wherein said pressure detection means comprise at least one pressure sensor associated with said tubular body in the proximity of said second end, where said pressure sensor is arranged inside said main channel.

2. The cannula according to claim 1, wherein said pressure sensor is of the type of a pressure transducer.

3. The cannula according to claim 1, wherein said first and second ends define at least a first mouth and at least a second mouth respectively, for the passage of the blood, and wherein said pressure sensor is arranged at said at least a second mouth.

4. The cannula according to claim 1, wherein said pressure detection means comprise electrical connecting means of said pressure sensor to pressure reading means and wherein said tubular body comprises at least one connecting channel, separate from said main channel and adapted to contain said electrical connecting means.

5. The cannula according to claim 4, wherein said connecting channel is defined on said tubular body and wherein said pressure sensor is arranged outside said connecting channel, on said tubular body a hole being defined for the passage of the connecting means which protrude from the connecting channel itself.

6. The cannula according to claim 5, wherein said tubular body is defined in a single body piece and wherein it comprises a plurality of said second mouths defined at said second end, said pressure sensor being located in the proximity of at least one of said second mouths.

7. The cannula according to claim 1, wherein said tubular body comprises at least one main body made of a flexible material and at least one connector associated with said main body and defining said second end, and wherein said pressure sensor is associated with said connector.

8. The cannula according to claim 7, wherein said pressure sensor is fixed to the inner wall of said connector delimiting said main channel.

9. The cannula according to claim 8, wherein said connecting channel is defined on said main body and by the fact that said hole is defined on said connector.

10. A cannula for drainage of blood vessels, the cannula comprising:
a tubular body including:
a first end,
a second end,
at least one main channel extending between said first end and said second end, wherein
said tubular body being movable between a home configuration and at least one extended configuration, in which the diameter of said main channel is reduced with respect to the home configuration;
elastic means associated with said tubular body and configured to or adapted to counteract the displacement from the home configuration to the extended configuration; and
pressure detection means configured for or programmed for detecting pressure, wherein
said pressure detection means includes at least one pressure sensor associated with said tubular body in the proximity of said second end, where said pressure sensor is arranged inside said main channel.

11. The cannula according to claim 10, wherein
said pressure sensor is of the type of a pressure transducer.

12. The cannula according to claim 10, wherein
said first and second ends define at least a first mouth and at least a second mouth respectively, for the passage of blood, and
said pressure sensor is arranged at said at least a second mouth.

12. The cannula according to claim 10, wherein
said pressure detection means includes electrical connecting means of said pressure sensor to pressure reading means and
said tubular body includes at least one connecting channel, separate from said main channel and adapted to contain said electrical connecting means.

13. The cannula according to claim 12, wherein said connecting channel is defined on said tubular body and wherein said pressure sensor is arranged outside said connecting channel, on said tubular body a hole being defined for the passage of the connecting means which protrude from the connecting channel itself.

14. The cannula according to claim 13, wherein
said tubular body is defined in a single body piece,
said tubular body includes a plurality of said second mouths defined at said second end, and
said pressure sensor being located in the proximity of at least one of said second mouths.

15. The cannula according to claim 10, wherein
said tubular body includes at least one main body made of a flexible material and at least one connector associated with said main body and defining said second end, and
said pressure sensor is associated with said connector.

16. The cannula according to claim 15, wherein
said pressure sensor is fixed to the inner wall of said connector delimiting said main channel.

17. The cannula according to claim 16, wherein
said connecting channel is defined on said main body and by the fact that said hole is defined on said connector.
